(19) United States
(12) Patent Application Publication
(10) Pub. No.: US 2024/0352668 A1
(43) Pub. Date: Oct. 24, 2024

(54) THIN FILM

(71) Applicant: The University of Tokyo, Bunkyo-ku, Tokyo (JP)

(72) Inventors: Takao Someya, Bunkyo-ku, Tokyo (JP); Yan Wang, Bunkyo-Ku, Tokyo (JP); Sunghoon Lee, Bunkyo-Ku, Tokyo (JP); Tomoyuki Yokota, Bunkyo-Ku, Tokyo (JP)

(21) Appl. No.: 18/688,528

(22) PCT Filed: Sep. 6, 2022
(86) PCT No.: PCT/JP2022/033352

§ 371 Date: Mar. 1, 2024

(30) Foreign Application Priority Data
Sep. 6, 2021 (JP) 2021-144778

(51) Int. Cl. D06N 3/12; A61B 5/27; A61B 5/28; D04H 1/4358; D04H 1/728; D06M 11/83; D06M 101/38; D06N 3/00

(52) U.S. Cl. CPC D06N 3/128; A61B 5/27; A61B 5/28; D04H 1/4358; D04H 1/728; D06M 11/83; D06N 3/0011; D06N 3/0015; D06M 2101/38; D06N 2201/0281; D10B 2509/00

(57) ABSTRACT

A thin film may include a high toughness and a high air permeability, e.g., when applied to skin, and may be used as a functional member or an electronic functional member. The thin film may include fibers, which may be formed into a net shape constituting a fibrous net, and a coating film, which may be formed on the surfaces of the fibers and in gaps between the fibers. The fibers may be formed by an electrospinning method. The fibers may be formed by using any of a polyurethane, a polyvinyl alcohol (PVA) derivative, and a polyvinylidene fluoride (PVDF) as a material. The coating film may be formed by using polydimethylsiloxane (PDMS) as a material. A conductive film, which may be formed on a part of all of the coating film, may also be provided.

### TECHNICAL FIELD

The present invention relates to a thin film, and for example, relates to a thin film that can be used as a functional member or an electronic functional member.

### BACKGROUND ART

In recent years, flexible electronics has attracted a lot of attention since softness of materials provides various applications. Particularly, in accordance with the worldwide aging of the society, the health care field has been drawing increasing attention. For example, attention has been paid as means for directly obtaining biological information from cells and tissues by being attached to the surface of a human body and inside the body.

In general, the flexible electronics is prepared by forming an electronics device on a flexible substrate. The flexible substrate is required to have high toughness since skin repeats expansion and contraction due to movement of the body or the like when being pasted to the skin to acquire biological information. On the other hand, if a thickness of the substrate is increased for the purpose of enhancing the toughness, the sense of discomfort at the time of attachment increases, and an air permeability of the substrate decreases, so that inflammation to the skin easily occurs, which is disadvantage. In addition, in the case of attachment to the skin, an adhesive layer for close contact with the skin is required, but a problem of the inflammation to the skin occurs due to components contained in the adhesive layer to be used in many cases.

In order to solve such a problem, there is proposed an electronic functional member having sufficiently high gas and moisture permeabilities by forming a fiber network of a nanofibers made of water-soluble polyvinyl alcohol (PVA) by an electrospinning method and depositing gold thereon to form an electrode layer (see, for example, Non Patent Literature 1).

In addition, there is proposed an electrode having high stretch resistance obtained by forming a fiber network of nanofibers made of polyurethane by an electrospinning method, creating a thin PDMS layer on the surface of the formed fiber network by a dip coating method, and depositing gold thereon (see, for example, Patent Literature 1).

In addition, efforts have also been made to form PDMS, which is a material having high affinity to skin and high adhesion, to a film thickness of 1 μm or less and use the PDMS as a soft substrate that is brought into close contact with a living body (see, for example, Non Patent Literature 2).

In addition, these soft substrates that are brought into close contact with a living body are not limited to the flexible electronics used to acquire biological information, and may be used for beauty care, treatment near a skin surface, protection of a wound site, and the like.

### CITATION LIST

### Patent Literature

Patent Literature 1: WO 2020/204171 A

### Non Patent Literature

Non Patent Literature 1: Akihito Miyamoto, et. al., Nature Nanotechnology 12, 907 (2017)

Non Patent Literature 2: Yamagishi, Kento, et al. “Tissue-adhesive wirelessly powered optoelectronic device for metronomic photodynamic cancer therapy.” Nature biomedical engineering 3.1 (2019): 27-36.

### SUMMARY OF INVENTION

### Technical Problem

It is desirable that a substrate used in close contact with a living body has high toughness and a high air permeability when being attached to skin. However, conventional functional members, electronic functional members, and the like have room for improvement in terms of the toughness and air permeability.

The invention has been made in view of the above points, and an object of the invention is to provide a thin film that has high toughness and a high air permeability when being attached to skin, and can be used as a functional member or an electronic functional member. In addition, another object thereof is to provide a functional member for beauty care or medical use excellent in adhesion to skin, air permeability, and toughness as well as an electrical functional member substrate and an electronic functional member.

### Solution to Problem

In order to achieve the objects described above, a thin film of the invention includes: a fiber formed in a mesh shape and forming a fiber network; and a coating film formed on the surface of the fiber and in an inter-fiber void. According to a preferred embodiment of the thin film of the invention, the fiber is formed by an electrospinning method. Here, the fiber may be made of any one of polyurethane, a polyvinyl alcohol (PVA) derivative, and polyvinylidene fluoride (PVDF) as a material, and the coating film may be made of polydimethylsiloxane (PDMS) as a material. Further, a conductive film formed entirely or partially on the coating film may also be provided.

In addition, according to another preferred embodiment of the thin film of the invention, an occupancy of the fiber is 5 to 50%, an occupancy of the coating film is 50% to 100%, and more preferably, the occupancy of the fiber is 10 to 30%.

### Advantageous Effects of Invention

The thin film of the invention that can be used as the functional member or the electronic functional member has high toughness and a high air permeability when being attached to skin.

### BRIEF DESCRIPTION OF DRAWINGS

FIG. 1 is a schematic view for describing a functional member according to a first embodiment of the invention.

FIG. 2 is a schematic view for describing an electronic functional member according to a second embodiment of the invention.

FIG. 3 is an electron micrograph of a conductive film of the electronic functional member according to the second embodiment of the invention.

FIG. 4 is a view showing water vapor permeabilities of the functional member according to the first embodiment of the invention and the electronic functional member according to the second embodiment of the invention.

FIG. 5 is a photograph showing an evaluation result of water resistance when the electronic functional member is attached to an arm.

FIG. 6 is a view showing results of an electrocardiogram measured by attaching the electronic functional member as an electrode to two positions of a wrist and an ankle.

FIG. 7 is a photograph showing evaluation results of water resistance of Comparative Example.

### DESCRIPTION OF EMBODIMENTS

Hereinafter, embodiments of the invention will be described with reference to the drawings, but a shape, a size, and an arrangement relationship of each constituent element are merely schematically illustrated to such an extent that the invention can be understood. In addition, a preferred configuration example of the invention will be described hereinafter, but a material, a numerical condition, and the like of each constituent element are merely preferred examples. Therefore, the invention is not limited to the following embodiments, and many modifications or variations that can achieve the effects of the invention can be made without departing from the scope of the configuration of the invention.

### Functional Member

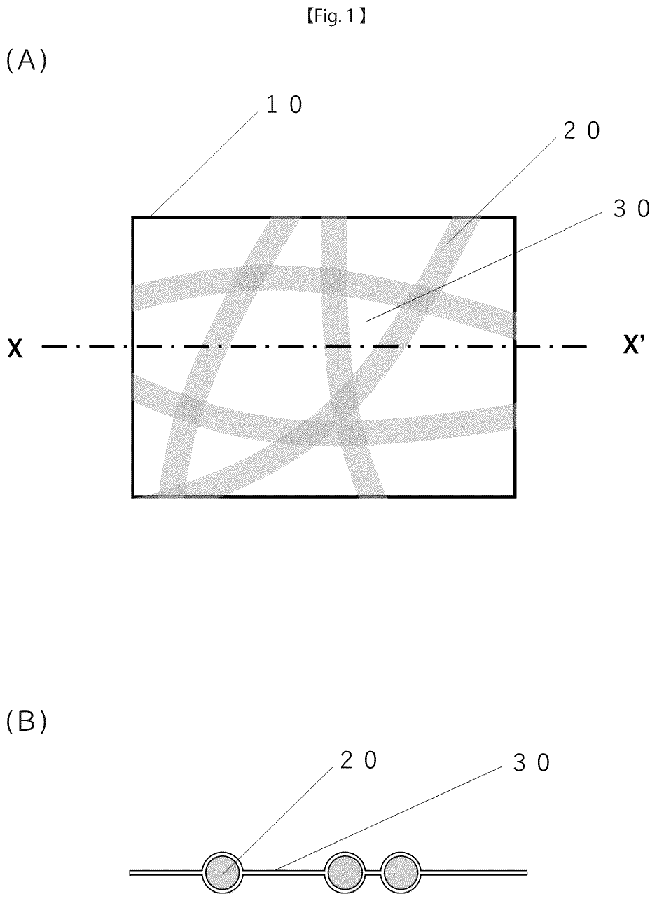

A functional member will be described as a first embodiment of a thin film of the invention with reference to FIG. 1. FIG. 1 is a schematic view for describing the functional member according to the first embodiment of the invention. FIG. 1(A) is a schematic top view of the functional member, and FIG. 1(B) is a schematic cross-sectional view of the functional member taken along line X-X′ in FIG. 1(A).

A functional member 10 includes a fiber 20 provided in a mesh shape and a coating film 30 coating the surface of the fiber 20 and a gap of a fiber network. As shown in FIG. 1(B), the coating film 30 exists as a continuous film so as to bury the surfaces of the fibers 20 and voids between the fibers 20.

Polyurethane is used as a core material of the fiber 20. In this case, the fiber network 10 is formed by an electrospinning method using a polyurethane solution of 13% by weight. As a solvent of this polyurethane solution, for example, a 7:3 mixed solution of N,N-dimethylformamide and methyl ethyl ketone is used. In addition, in forming the fiber network 10, conditions of the electrospinning method may be a spinning time of 20 minutes, an applied voltage of 25 kV, and a discharge rate of 1 ml/hour. After the electrospinning method is performed, ultraviolet (UV) ozone treatment is performed for 1 minute to obtain the fiber network 10.

Note that a diameter of the fiber 20, in this example, a polyurethane fiber is preferably in a range of 100 nm to 1 μm, and more preferably in a range of 200 nm to 700 nm. In this case, the polyurethane fiber is a so-called nanofiber. In addition, a Young's modulus of polyurethane is 100 MPa to 700 MPa, and the Young's modulus is relatively high.

Although the example in which polyurethane is used as the core material has been described here, the invention is not limited thereto. The core material may be any material that can form a fiber network of nanofibers by an electrospinning method. This is because it is difficult to form a fiber network of nanofibers with a material having an extremely low Young's modulus. As the core material, for example, a polyvinyl alcohol (PVA) derivative, polyvinylidene fluoride (PVDF), or the like can be used in addition to polyurethane.

As a coating material forming the coating film 30, a silicone resin, for example, polydimethylsiloxane (PDMS) is used. In this case, the coating film 30 is formed by applying dip coating using a PDMS solution to the fiber. This PDMS solution is obtained by dissolving a PDMS precursor in hexane. A weight ratio of the PDMS precursor to hexane is, for example, 1:30.

A film thickness of the coating film 30 is preferably in a range of 30 nm to 300 nm, and more preferably in a range of 50 nm to 150 nm. In addition, a Young's modulus of PDMS is generally 4 MPa to 40 MPa, and the Young's modulus is relatively low.

Although the example in which PDMS is used as the coating material has been described here, the invention is not limited thereto. The coating material is preferably a material having a low Young's modulus. As the coating material, for example, an ethylene vinyl acetate (EVA) resin or the like can be used in addition to PDMS.

### Electronic Functional Member

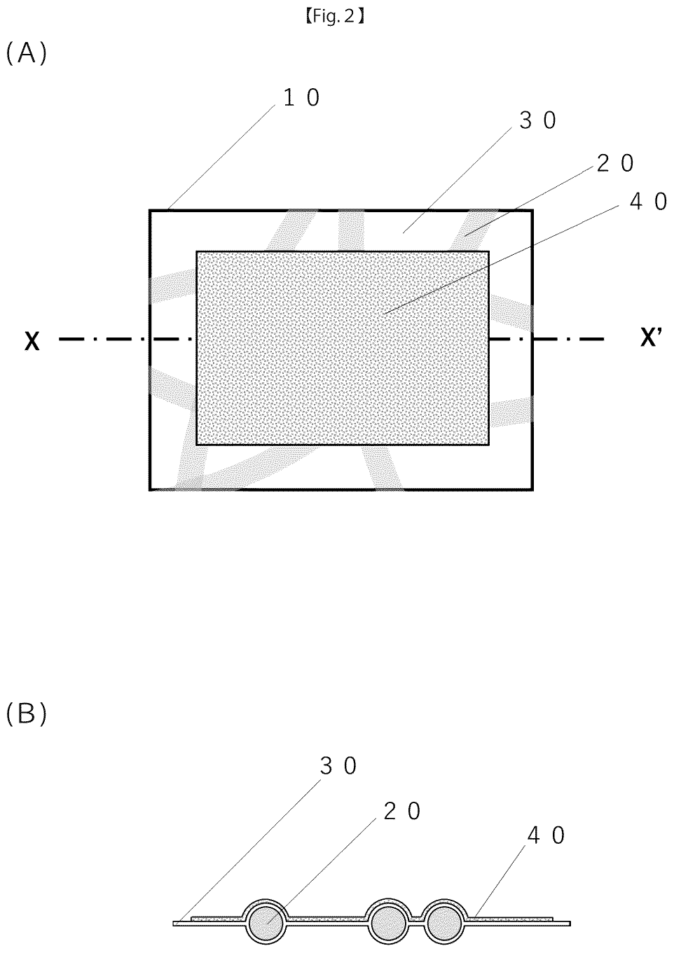

An electronic functional member will be described as a second embodiment of the thin film of the invention with reference to FIG. 2. FIG. 2 is a schematic view for describing the electronic functional member according to the second embodiment of the invention. FIG. 2(A) is a schematic top view of the electronic functional member, and FIG. 2(B) is a schematic cross-sectional view of the electronic functional member taken along line X-X′in FIG. 2(A).

The electronic functional member has a configuration in which a conductive film 40 is formed on the entire surface or a part of the surface of the functional member according to the first embodiment of the invention described above. Gold (Au) is used as a conductive member forming the conductive film 40. In this case, the conductive film 40 is formed by vacuum deposition of Au on the functional member.

A film thickness of the conductive film 40 is preferably in a range of 30 nm to 300 nm, and more preferably 50 nm to 150 nm.

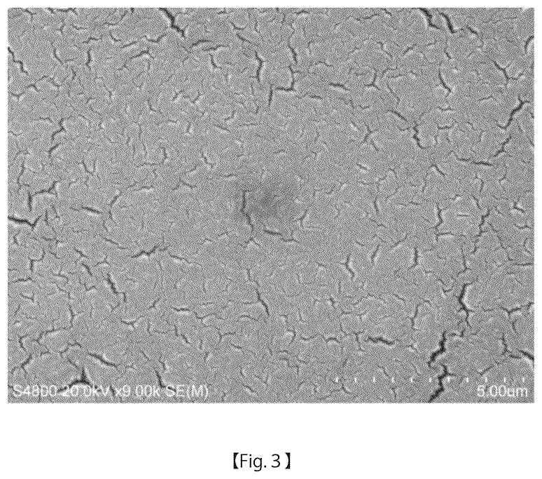

FIG. 3 is an electron micrograph of a conductive film of the electronic functional member according to the second embodiment of the invention. As shown in FIG. 3, cracks (black regions in the photograph) are generated in some places of the conductive film, and there is a region where Au does not exist. The presence of cracks is largely related to a water vapor permeability of the electronic functional member, which will be described later.

Although the example in which Au is used as the conductive member forming the conductive film 40 has been described, the invention is not limited thereto. As the conductive member, silver (Ag), titanium (Ti), platinum (Pt), or the like can be used in addition to Au. In addition, a method for forming the conductive film is not limited to vacuum deposition. As the method for forming the conductive film, a sputtering method, spin coating using a dispersion liquid of the conductive member, slit coating, or screen printing can be used.

### Evaluation of Toughness of Functional Member

Evaluation of toughness of the functional member according to the first embodiment of the invention will be described. Table 1 is a table showing results of toughness evaluation for a functional member as Example and a PDMS film having a thickness of 720 nm as Comparative Example.

| Evaluation Sample | Toughness(J/m³) |
|---|---|
| Example (functional member) | 3.4 |
| Comparative Example (PDMS film) | 0.6 |

In the functional member of Example used in this evaluation, polyurethane was used as the fiber network 20 at a density of 0.36 mg/cm². In addition, a film thickness of the coating film 30 was 95 nm.

The PDMS film having the thickness of 720 nm used as Comparative Example was prepared by the following method. First, a glass substrate was coated with polytetrafluoroethylene (PTFE) as a release layer, and an aqueous PVA solution of 10% by weight was applied thereon and dried to form a PVA thin film. Thereafter, PDMS of 6% by weight dissolved in hexane was applied on the PVA thin film by spin coating and dried. Thereafter, the PDMS film/PVA layer was peeled off from the glass substrate, and then, the PVA layer was dissolved in water and removed to prepare the PDMS film having the film thickness of 720 nm. The film thickness of 720 nm was achieved by controlling the number of revolutions of a spinner in the spin coating of the PDMS of 6% by weight dissolved in hexane.

The toughness evaluation was performed using AG-X manufactured by Shimadzu Corporation. As shown in Table 1, toughness of the functional member was 3.4 J/m³. On the other hand, toughness of the PDMS film having the thickness of 720 nm used as Comparative Example was 0.6 J/m³.

As described above, the toughness of the functional member including the coating film having the thickness of 95 nm was five or more times a value of the toughness of the PDMS film having the thickness of 720 nm.

Such a result is attributed to the fact that the functional member has a polyurethane fiber network in the form of reinforcing the PDMS film, whereas the toughness is determined by the material itself of PDMS in the PDMS film having the thickness of 720 nm used as Comparative Example. As described above, when the thin film of the invention includes the polyurethane fiber network, higher toughness can be obtained with a thinner film thickness.

### Evaluation of Water Vapor Permeability

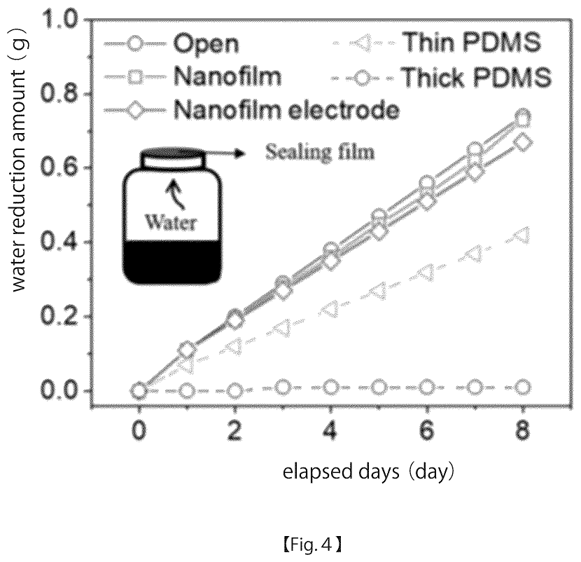

With reference to FIG. 4, water vapor permeability evaluation for the functional member according to the first embodiment of the invention and the functional member according to the second embodiment of the invention will be described. FIG. 4 is a view showing water vapor permeabilities of the functional member according to the first embodiment of the invention and the electronic functional member according to the second embodiment of the invention.

Regarding the water vapor permeability evaluation, the evaluation was performed by comparing a water reduction amount in a case where water was poured into a glass bottle and the top of the glass bottle was open with water reduction amounts in a case where the top of the glass bottle was sealed with the functional member, the electronic functional member, and a PDMS film having a film thickness of 1 μm and a PDMS film having a film thickness of 1.4 mm used as Comparative Examples. Since the water poured in the glass bottle evaporates from a mouth of the bottle as water vapor, a larger water reduction amount means a higher water vapor permeability. In FIG. 4, the horizontal axis represents elapsed days (unit: day), and the vertical axis represents a water reduction amount (unit: g).

A value (Open) plotted by a circle mark and a solid line indicates a water reduction amount when the top of the glass bottle is open; a value (Nanofilm) plotted by a square mark and a solid line indicates a water reduction amount when the top of the glass bottle is sealed using the functional member; a value (Nanofilm electrode) plotted by a diamond mark and a solid line indicates a water reduction amount when the top of the glass bottle is sealed using the electronic functional member; a value (Thin PDMS) plotted by a triangle mark and a broken line indicates the water reduction amount when the top of the glass bottle is sealed using the PDMS having the film thickness of 1 μm; and a value (Thick PDMS) plotted by a circle mark and a broken line indicates a water reduction amount when the top of the glass bottle is sealed using the PDMS having the film thickness of 1.4 mm.

The configuration of the functional member is the same as that of the above-described functional member evaluated for toughness. In addition, the electronic functional member is formed by depositing Au with a thickness of 70 nm on the above-described functional member evaluated for toughness by vacuum deposition. The PDMS having the thickness of 1 μm was obtained by preparing the PDMS film used in the toughness evaluation described above by the same method as in Comparative Example when the toughness was evaluated, and obtaining the thickness of 1 μm by controlling the number of revolutions in spin coating of PDMS of 6% by weight dissolved in hexane. In addition, a commercially available PDMS film was used as the PDMS having the film thickness of 1.4 mm.

As can be seen from the results of FIG. 4, the functional member and the electronic functional member show the water reduction amounts similar to that in a case where the top of the glass bottle is open, and it can be seen that the water vapor permeabilities are high. It is considered that the water vapor permeability of the functional member is high since the PDMS film is as extremely thin as 95 nm. The reason why the water vapor permeability is also high in the electronic functional member on which Au is deposited is considered to be that a part of the deposited Au is cracked as shown in FIG. 3 so that water vapor diffuses from the cracked part.

On the other hand, a water vapor permeability of the PDMS having the film thickness of 1 μm showed a low value because the film thickness of the PDMS was larger than that of the functional member by about one order of magnitude. In addition, it can be seen that the PDMS having the film thickness of 1.4 mm has almost no water vapor permeability because the film thickness of the PDMS is thicker than that of the electronic functional member substrate by four orders of magnitude or more.

### Adhesion Evaluation of Functional Member and Electronic Functional Member

With reference to Table 2, evaluation of adhesion to skin of each of a functional member and an electronic functional member will be described. Table 2 is a table showing results of adhesion evaluation for the functional member, the electronic functional member, and Comparative Examples. The adhesion evaluation was performed by bringing each sample into close contact with a bio skin plate (product number: P001-001) which is artificial skin manufactured by Beaulax Co., Ltd. An adhesive force was evaluated using AG-X manufactured by Shimadzu Corporation.

| Evaluation Sample | Adhesion (μJ/cm²) |
|---|---|
| Example 1 | 158 |
| Example 2 | 62 |
| Example 3 | 20 |
| Comparative Example 1 | 8.0 |
| Comparative Example 2 | 0 |

In Example 1, a thin film having the same configuration as the functional member used in the water vapor permeability evaluation described above was used. In Examples 2 and 3, a thin film having the same configuration as the electronic functional member used in the water vapor permeability evaluation described above was used. In Example 2, a surface of the electronic functional member on which the PDMS is exposed (the surface on which the conductive film of Au is not formed) is brought into close contact with the artificial skin. In addition, in Example 3, a surface of the electronic functional member on which the conductive film of Au is formed is brought into close contact with the artificial skin.

In Comparative Example 1, the PDMS film having the thickness of 720 nm used in the toughness evaluation described above was brought into close contact with the artificial skin. In addition, in Comparative Example 2, a nanomesh electrode, disclosed in Patent Literature 1 and obtained by coating a surface of a polyurethane fiber network with PDMS and depositing Au thereon, is brought into close contact with the artificial skin. A density of the polyurethane fiber network was 0.36 mg/cm², a film thickness of the PDMS applied on the surface of polyurethane was 200 nm, and a film thickness of the vacuum-deposited Au was 70 nm. The vacuum deposition of Au was performed from both sides on the surface and a back surface of the polyurethane fiber network.

An adhesive force when the functional member of Example 1 is brought into close contact with the artificial skin is 158 μJ/cm², which is the largest, an adhesive force when the surface of the electronic functional member of Example 2 where the PDMS is exposed is brought into close contact with the artificial skin is 62 μJ/cm², which is the second largest, and an adhesive force when the surface of the electronic functional member of Example 3 where the conductive film of Au is formed is brought into close contact with the artificial skin is 20 μJ/cm², which is the third largest. An adhesive force of Comparative Example 1 was 8.0 μJ/cm², and an adhesive force of Comparative Example 2 was 0 μJ/cm².

The reason why the adhesive force of Example 1 is larger than that of Example 2 is considered to be that Example 2 is obtained by forming a metal thin film on the surface of Example 1, and thus, has lower flexibility than that of Example 1, and the followability to the artificial skin is slightly inferior to that of Example 1.

In addition, the reason why the adhesive force of Example 2 is larger than that of Example 3 is considered to be that an adhesive force of the PDMS at an interface with the artificial skin is higher than that of Au since the surface in contact with the artificial skin is Au in Example 3 while the surface in contact with the artificial skin is PDMS in Example 2. In addition, the reason why all of Examples 1, 2, and 3 have higher adhesive forces than that of Comparative Example 1 is considered to be that the functional member and the electronic functional member follow and come into contact with fine irregularities of the artificial skin since the film thicknesses of the PDMS coating films of Examples 1, 2, and 3 are thinner than the film thickness of the PMDS of Comparative Example 1 by about one order of magnitude. In addition, the reason why there is no adhesive force of the nanomesh electrode described in Patent Literature 1 is considered to be that the adhesive force of the deposited Au to the artificial skin is poor in addition to the small area of contact between the nanomesh electrode and the artificial skin. It is a well-known fact that, in a case where a sheet-like device is pasted to skin, a peeling force is easily applied to a peripheral part of the sheet. In addition, the adhesive force to the skin is the highest in the functional member shown in Example 1 as apparent from Table 2. Accordingly, it is apparent that, when an electrical functional member shown in Example 2 or Example 3 on which the electrode is formed is pasted to the skin, the electrical functional member is hardly peeled off from the skin by adopting a configuration in which no electrode region is provided in a peripheral portion as shown in FIG. 2(A). That is, the conductive film is not necessarily formed on the entire coating film, and the invention also encompasses a configuration in which the conductive film is formed in a partial region on the coating film.

Table 3 shows results of adhesion evaluation in a case where a coating film in which some of gaps of a fiber network exist as voids is formed, Au is deposited thereon, and a surface on which Au is deposited is pasted to skin with the configuration of Example 3 and the method of adhesion to the skin.

| Evaluation Sample | Occupancy (%) | Adhesion (μJ/cm²) |
|---|---|---|
| Sample 1 | 100 | 20 |
| Sample 2 | 80 | 15 |
| Sample 3 | 50 | 11 |
| Sample 4 | 30 | 5 |

Sample 1 is the same sample as that of Example 3 shown in Table 2, and Samples 2 to 4 show evaluation results of adhesive forces of samples having different occupancies (coverages) of the coating film with respect to the functional member. The adhesive force increases as the coverage increases. When the coverage was 50%, the adhesive force was 11 μJ/cm², which was higher than those of Comparative Examples in Table 2. Therefore, the coating film is not necessarily formed as a continuous film having the occupancy of 100%. In a case where the coating film is not the continuous film, the coverage is preferably 50% or more in order to exhibit a more excellent adhesive force than those of Comparative Examples. Note that the coverage was controlled by adjusting a weight ratio of a PDMS precursor to hexane at the time of forming the coating film.

Here, the coverage, which is the occupancy of the coating film, is a proportion of the area occupied by the coating film per unit area. As described above, the coverage is preferably 50% to 100%. At this time, the occupancy of fibers is preferably 5% to 50%, and is a value lower than the coverage. Note that there is a possibility that toughness decreases when the occupancy of fibers is low, and thus, the occupancy of fibers is more preferably 10% to 30%.

### Evaluation of Water Resistance of Electronic Functional Member

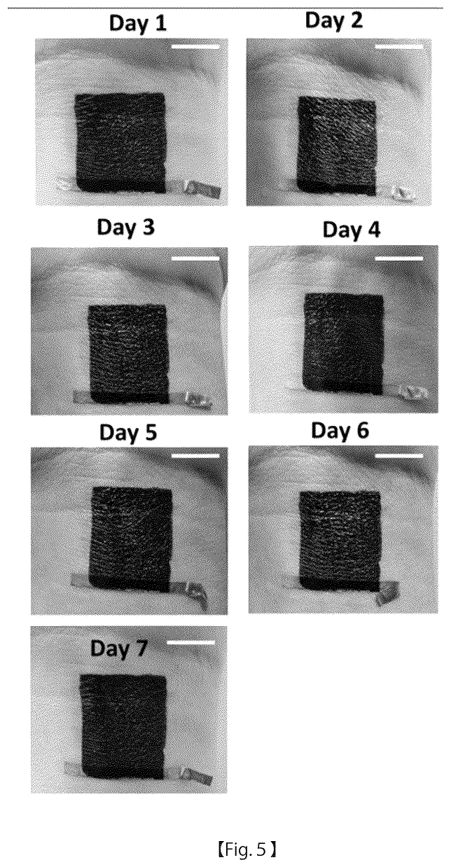

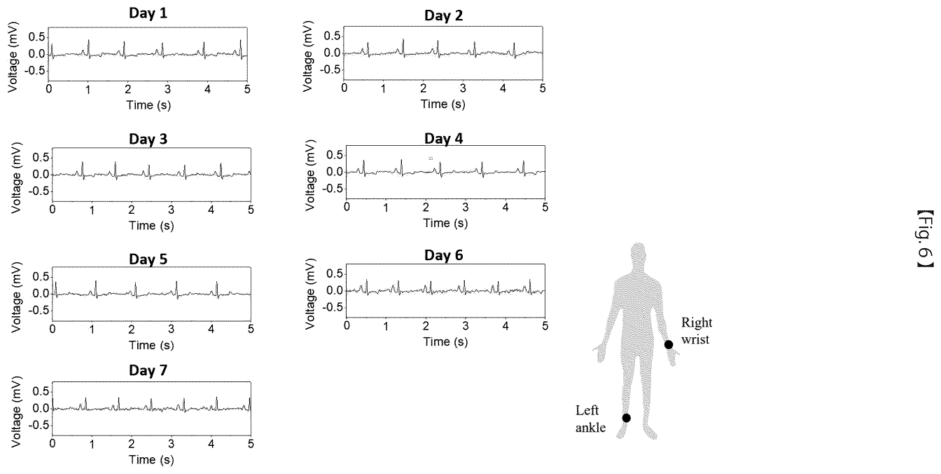

Evaluation results of water resistance of an electronic functional member will be described with reference to FIGS. 5 and 6. FIG. 5 is a photograph showing evaluation results of water resistance when the electronic functional member is attached to an arm. In addition, FIG. 6 is a view showing results of an electrocardiogram measured by attaching the electronic functional member as an electrode to two positions of a wrist and an ankle. As the electronic functional member used in this evaluation, an electronic functional member having the same configuration as that of Example 3 used in the adhesive force evaluation of Table 2 was used.

FIG. 5 shows a change in the electronic functional member attached to the arm for seven days, and it has been found that there is no change in adhesion to skin in spite of a daily shower. In addition, it could be confirmed that an electronic mechanism member functions as the electrode since the electrocardiogram could be stably measured for seven days as shown in FIG. 6.

In addition, it has been also confirmed that there is no change in the skin condition after use, and there is no skin sensitization property due to the attachment of this electronic functional member.

When a surface of an electrical functional member on which a conductive film is formed is brought into close contact with skin and used as an electrode, a conductive film formed on a film can be sandwiched between the skin and the electrical functional member in a configuration in which this conductive film is in contact with the conductive film of the electrical functional member to take the electrode out to the outside of the electrical functional member. This enables electrical connection to an external measurement circuit in electrocardiographic measurement or the like. The electrical functional member of the invention itself may be used as the conductive film formed on the film.

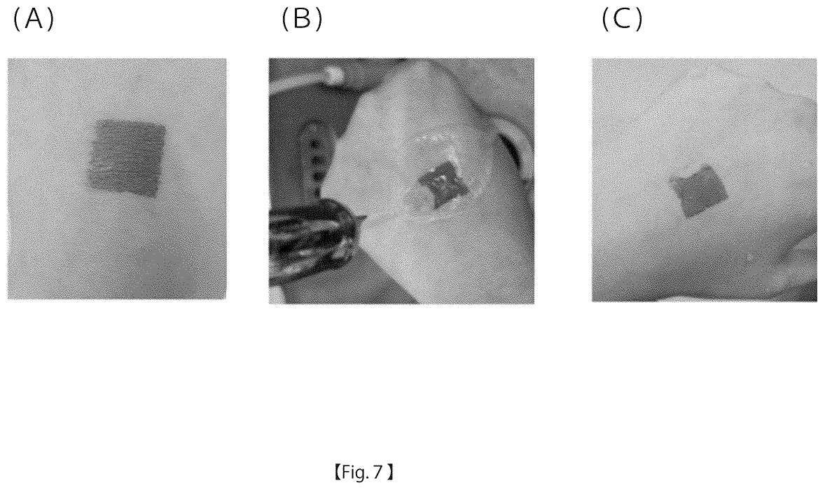

FIG. 7 is a photograph showing evaluation results of water resistance of the nanomesh electrode used as Comparative Example of the adhesion evaluation described above. FIG. 7(A) shows a state in which the nanomesh electrode of Comparative Example is attached to skin, FIG. 7(B) shows a state in which a water flow is applied to the nanomesh electrode, and FIG. 7(C) shows the nanomesh electrode after the water flow is applied. Since the nanomesh electrode used here has no adhesion to the skin, an aqueous solution of PVA was applied to the skin surface in advance, and the nanomesh electrode was pasted to be in close contact with the skin before the aqueous solution was dried. It was found that a part of the electrode was peeled off only by applying the water flow to the nanomesh electrode, and it has been confirmed that the water resistance was poor.

It has been also confirmed that moistening the skin surface with water spray or the like in advance before bringing the functional member or the electronic functional member of the invention into close contact with the skin makes it possible to remove an air layer existing between the skin surface and the functional member or the electronic functional member, and has an effect of enhancing an adhesive force to the skin.

### REFERENCE SIGNS LIST

10 Functional member
20 Fiber
30 Coating film
40 Conductive film

1. A thin film comprising:
a fiber formed in a mesh shape and forming a fiber network; and
a coating film formed on a surface of the fiber and in an inter-fiber void.

2. The thin film of claim 1, wherein the fiber is formed by an electrospinning method.

3. The thin film of claim 1, wherein:
the fiber is made of any one of polyurethane, a polyvinyl alcohol (PVA) derivative, and polyvinylidene fluoride (PVDF) as a material, and
the coating film is made of polydimethylsiloxane (PDMS) as a material.

4. The thin film of claim 1, further comprising a conductive film formed entirely or partially on the coating film.

5. The thin film of claim 1, wherein:
an occupancy of the fiber is 5 to 50%, and
an occupancy of the coating film is 50% to 100%.

6. The thin film of claim 5, wherein the occupancy of the fiber is 10 to 30%.
